(19) United States
(12) Patent Application Publication
(10) Pub. No.: US 2006/0094872 A1
(43) Pub. Date: May 4, 2006

(54) Process for the preparation of cephalosporin antibiotic

(75) Inventors: Udayampalayam P. Senthilkumar, Chennai (IN); Kanagaraj Sureshkumar, Chennai (IN); Singaravel Mohan, Chennai (IN); Lakshminarayanan Arunkumar, Chennai (IN); Bakthavachalam Ananthan, Chennai (IN)

Correspondence Address: OLIFF & BERRIDGE, PLC, P.O. BOX 19928, ALEXANDRIA, VA, 22320, US

(73) Assignee: Orchid Chemicals & Pharmaceuticals Ltd., Chennai (IN)

(21) Appl. No.: 11/294,410

(22) Filed: Dec. 6, 2005

Related U.S. Application Data
(63) Continuation-in-part of application No. 10/922,991, filed on Aug. 23, 2004.

(30) Foreign Application Priority Data
Aug. 22, 2003 (IN) 673/CHE/2003

(51) Int. Cl. C07D 501/00

(52) U.S. Cl. 540/217

(57) ABSTRACT

An improved process for the preparation of Ceftiofar sodium of formula (I) without isolating intermediate compound of formula (IV)

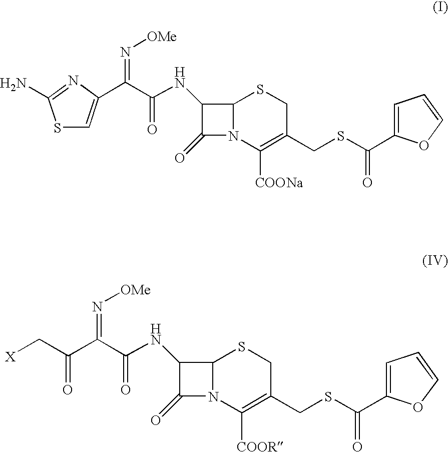

### CROSS-REFERENCE TO RELATED APPLICATION

This application is continuation in part application of our co-pending application Ser. No. 10/922,991, filed Aug. 23, 2004, which claims priority from 673/CHE/2003 filed on Aug. 22, 2003. The entire disclosures of the prior applications is incorporated herein by reference.

### BACKGROUND

The present invention relates to a process for the preparation of cephalosporin antibiotic of the formula (I), more particularly relates to preparation of Ceftiofur sodium of formula (I).

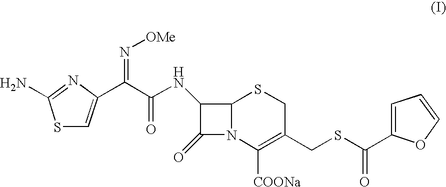

Ceftiofur, a semisynthetic cephalosporin, is a broad-spectrum antibiotic against both Gram-positive and Gram-negative bacteria including beta-lactamase-producing bacterial strains and anaerobes. Its antibacterial activity results from the inhibition of mucopeptide synthesis in the cell wall in a similar fashion to other cephalosporins. Ceftiofur is used in the treatment of respiratory infections in cattle and pigs. The chemical designation is [6R-[6a,7b(z)]]-7-[[(2-amino-4-thiazolyl)(methoxyimino)acetyl]amino]-3-[[2-furanylcarbonyl)thio]methyl]-8-oxo-5-thia-1-azabicyclo[4.2.0]oct-2-ene-2-carboxylic acid. The sodium and hydrochloride salts are administered intramuscularly and intravenously.

Ceftiofur is first disclosed in U.S. Pat. No. 4,464,367, which also discloses a process for preparing Ceftiofur and its sodium salt. U.S. Pat. No. 4,937,330 disclose a process for preparing Ceftiofur sodium, according to this patent Ceftiofur sodium was precipitated as solid from aqueous THF.

There are various literature methods reported for the preparation of cephalosporin compounds like Ceftiofur which are summarized below:

U.S. Pat. No. 5,109,131 describes a process in which 4-halo-2-methoxyimino-3-oxobutyric acid, is reacted with cephem moiety as per the scheme depicted below: 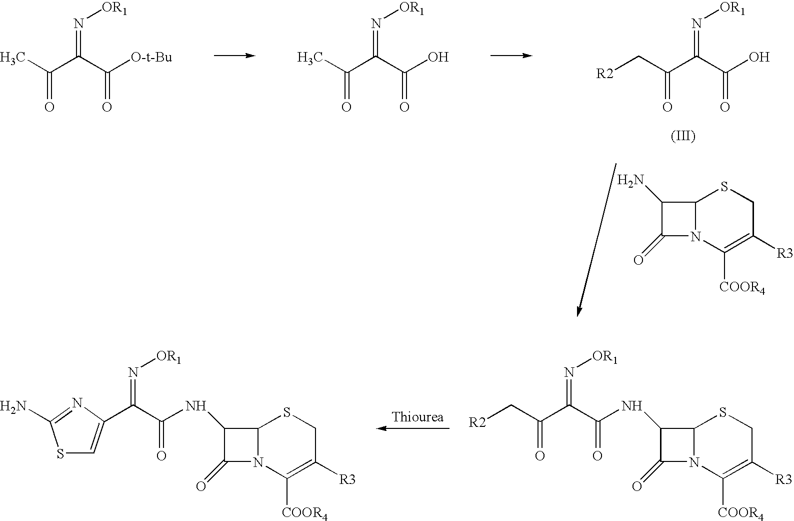
wherein R₁ stands for a C₁₋₄ alkyl group optionally substituted with carboxyl or a C₁₋₄ alkoxy-carbonyl group, R₂ stands for a halogen atom, R₃ stands for hydrogen atom or a standard cephalosporin substituent which includes Ceftiofur, and R₄ stands for hydrogen atom or a group which can be converted to hydrogen

U.S. Pat. No. 4,298,529 describes a similar process as depicted in U.S. Pat. No. 5,109,131, according to this patent the cephem compound of formula may be used as such or as a silyl derivative (column 12, lines 20-23 of U.S. Pat. No. 4,298,529).

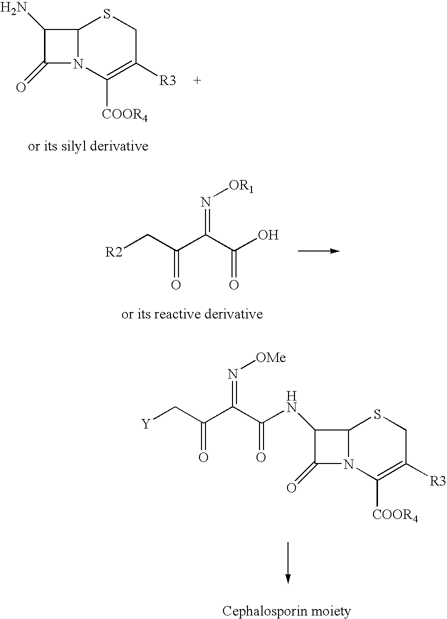

CA 1,146,165, also discloses a similar approach for the preparation of cephalosporin compounds.

EP 0030294 discloses a process for the preparation of compound of cephalosporin antibiotic as given in scheme 1:

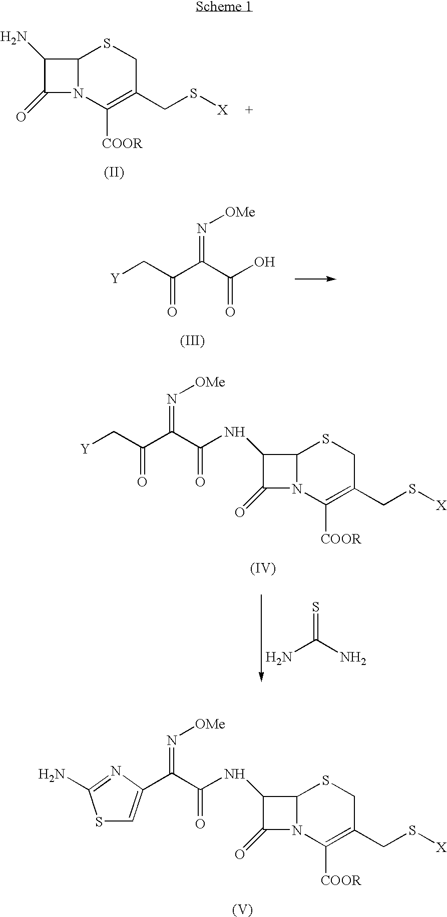

GB 2012276 describes 7-(4-halogeno-3-oxo-2-alkoxyiminobutyrylamino) cephalosporin derivative of the formula (XIII) 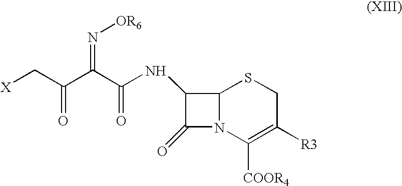
wherein X represents a halogen atom, R³ represents —CH₂R⁵ (R⁵ is hydrogen atom or the residue of a nucleophilic compound), a halogen atom, an alkoxyl group, thiol group, amino group etc., —COOR⁴ represents a carboxylic group that may be esterified, and R⁶ represents an alkyl group and also a process for preparing a 7-[2-(2-aminothiazol-4-yl)-2-(syn)-alkoxyiminoacetamido] cephalosporin derivatives of the formula (XIV)

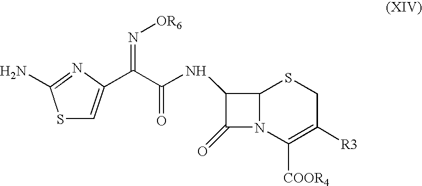

U.S. Pat. No. 6,552,186 relates to the preparation of ceftriaxone and cefotaxime also claims a process for the preparation of number of cephalosporin antibiotic including Ceftiofur using similar approach disclosed in prior art. As cited by U.S. Publication No. 2005/0059820, this patents itself obvious and anticipated over various prior art. Moreover this patent utilizes two phase solvent system for cyclization stage; one of the disadvantages with this two phase solvent system during cyclization with thiourea is that the reaction takes more times for completion or many times the reaction will not proceed for completion leaving 7% to 15% starting material, and affording less pure product.

Thus the above literature reports like CA 1,146,165, U.S. Pat. No. 4,298,529 and U.S. Pat. No. 5,109,131 (which were published after the grant of U.S. Pat. No. 4,464,367, where Ceftiofur was first disclosed) and U.S. Pat. No. 6,552,186 pertaining towards the preparation of Cephalosporin antibiotics suggest and teach the following general scheme for the preparation of Ceftioflir of formula (I):

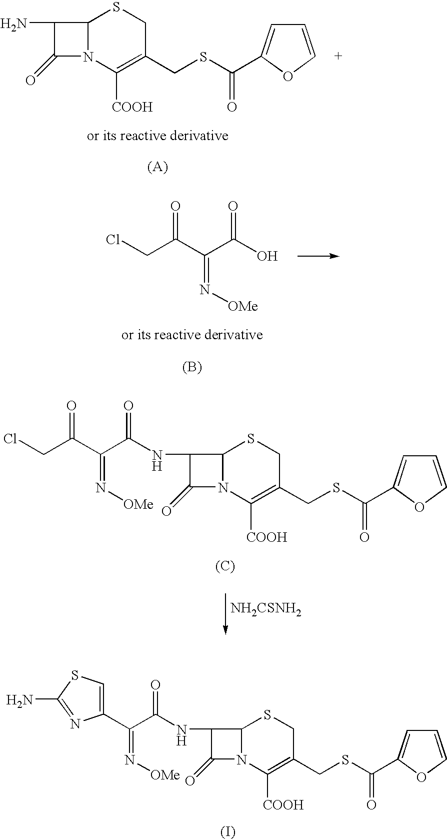

Though the literature pertains to cephalosporin chemistry, which suggests or motivates the above general process, U.S. Pat. No. 6,458,949 claims a similar process for preparing Ceftiofur. According to this patent the purity of final Ceftiofur depends critically on the isolation of compound of formula (C). Claim 7 of this patent reads that “starting” with compound of formula (C), which clearly indicates the isolation of formula (C) is crucial as per this patent. This patent also acknowledges that cyclization of compound of formula (C) in situ with thiourea in the presence of a base yields impure Ceftiofur and further purifications are difficult, time consuming and do not result in a product of good quality. Also this patent claims the compound of formula (C) though it is obvious over cephalosporin chemistry.

Interestingly, in our continued research we have identified a simple process for the preparation of Ceftiofur, in which even though the compound of formula (C) is not isolated, it yields Ceftiofur in highly pure form. The in situ process of this invention avoids the time-consuming isolation step of the intermediate and makes overall process commercially viable with reduced time-cycle and economical. None of the prior art suggests or even motivates the present invention.

### SUMMARY

An objective of the invention is to provide an improved process for the preparation of cephalosporin antibiotic of the formula (I), without isolating the compound of formula (IV).

Another objective of the present invention is to provide an improved process for the preparation of Ceftiofur sodium of the formula (I) in high purity and yield.

Accordingly, the present invention provides an improved process for the preparation of Ceftiofur sodium of the formula (I) 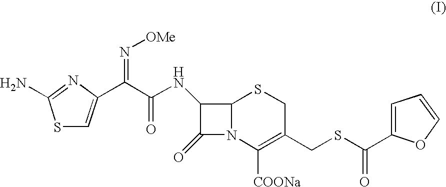
which comprises:
(i) activating the compound of formula (E) as acid chloride of formula (Ea) in an organic solvent 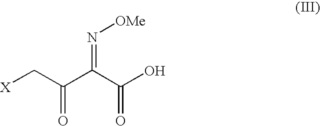
where X represents halogen atom such as chlorine or bromine, using a halogenating agent,
(ii) treating the reaction mass obtained from step (i) with water at a temperature in the range of −40° C. to 10° C.,
(iii) separating the organic layer containing the activated derivative of formula (IIIa) and condensing the activated derivative of the formula (IIIa) 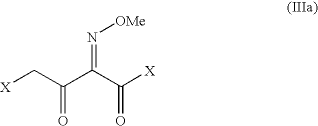
where X represents halogen atom such as chlorine or bromine, with 7-amino cephalosporin derivative (FURACA) of the formula (II) or its reactive derivative 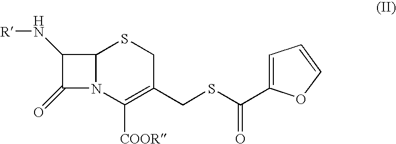
wherein R′ represents hydrogen, or silyl and R″ represents hydrogen or silyl in the presence of a solvent and in the presence or absence of base at a temperature in the range of −50° C. to 10° C. to produce a compound of formula (IV) 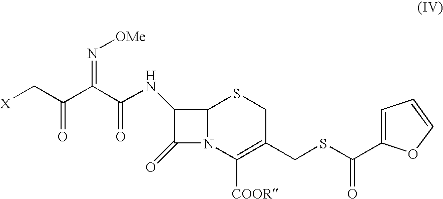
where all symbols are as defined above, and
iv) optionally removing the solvent of step (iii) reaction mass and cyclizing the compound of formula (IV) with thiourea, in water, in the presence or absence of water miscible solvent and sodium ion source, at a temperature in the range of −50 to 30° C. to produce compound of formula (I), wherein the improvement comprises producing the compound of formula (I) without isolating compound of formula (IV), and also characterized by one or more of the following improvements:
a) removing the solvent in step (iii),
b) conducting the reaction of step (iv) in a homogeneous solvent system.

The said process is depicted as below:

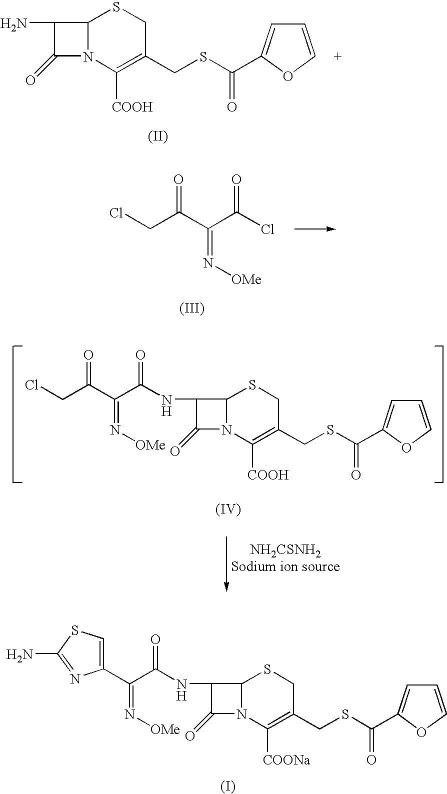

### DETAILED DESCRIPTION OF EMBODIMENTS

In an embodiment of the present invention the halogenating agent for activating the acid of formula (III) in step (i) is selected from PCl₅, PCl₃, POCl₃, SOCl₂ and the like, and the organic solvent employed in step (i) is selected from dichloromethane, ethyl acetate, THF, DMF and the like or any inert solvent can be employed.

In another embodiment of the present invention the treatment of step (i) reaction mass with water at low temperatures removes the impurities formed. Because of this treatment, Ceftiofur sodium was obtained in pure form even without isolating the compound of formula (IV). This constitutes one of the advantages of the present invention.

In still another embodiment of the present invention, the condensation of FURACA of formula (II) with (IIIa) is performed in the presence of a solvent selected from dichloromethane, ethyl acetate, methanol, ethanol, isopropanol, isobutyl alcohol, n-propanol, n-butanol, tert-butanol, tetrahydrofuran, aromatic hydrocarbons, acetone, ethyl methyl ketone, diethyl ketone, pentan-3-one, cyclohexanone, methyl isobutyl ketone, dioxane, acetonitrile, DMAc, N,N-dimethylformamide, dialkylethers, ethylene glycol, ethylene glycol monomethyl ether, diglyme, monoglyme, diethylene glycol, triethylene glycol, polyethylene glycol, water and the like or mixtures thereof.

In yet another embodiment of the present invention, the base used in step (iii) is selected from ammonia, sodium carbonate, sodium bicarbonate, ammonium carbonate, potassium carbonate, sodium hydroxide, potassium hydroxide, trimethyl amine and the like. The presence of base facilitates the condensation, when the compound of formula (II) is employed in free form. Accordingly the base is necessary when the compound of formula (II) is employed in free form and it is not essential when the compound of formula (II) is employed in the form of silylated derivative.

In yet another embodiment of the present invention, the compound of formula (IV) is prepared by condensing the reactive derivative of compound of formula (II), wherein the reactive derivate is silylated form of formula (II), with (IIIa). Silylated form of formula (II) is prepared by treating the compound of formula (II) with silylating agents like hexamethyldisilazane (HMDS), trimethylsilyl chloride (TMCS), bistrimethylsilyl urea (BSU), N,O-Bistrimethylsilyl acetamide (BSA) and the like in the presence or absence of catalyst like N-methyl morpholine, acetamide and imidazole. The solvent used for silylation and subsequent condensation is selected from dichloromethane, N,N-dimethylformamide, N,N-dimethylacetamide, acetonitrile, toluene and the like or mixtures thereof more particularly dichloromethane.

In another embodiment of the present invention the solvent employed for silylation and subsequent condensation can be removed by either distillation or by any conventional method so as to conduct the cyclization step in homogeneous solvent system. Conventional method involves quenching of this reaction mass to methanol or water. However, it has been observed the impurity formation in conventional method is high when compared to distillation, which is an advantage of the present invention. It has been also observed that the conventional two-phase solvent system takes more time for cyclization, and produces less pure Ceftiofur.

In still another embodiment of the present invention, the present invention was performed without isolating the compound of formula (IV), which makes the reaction as one pot, which is also considered to be one of the advantages of the present invention.

In yet another embodiment of the present invention, the cyclization of compound of (IV) is carried out using water miscible solvent selected from tetrahydrofuran, acetone, ethyl methyl ketone, methyl isobutyl ketone, methyl isopropyl ketone, cyclohexanone, diethyl ketone, pentan-3-one, cyclohexanone, acetonitrile, N,N-dimethylformamide, N,N-dimethylacetamide, dioxane, (C₁-C₅)alcohol, ethylene glycol, diglyme, monoglyme, ethylene glycol monomethyl ether, diethylene glycol, triethylene glycol, polyethylene glycol and the like or mixtures there of, and sodium ion source employed in step (iv) is selected from sodium acetate, sodium carbonate, sodium bicarbonate, sodium methoxide, sodium 2-ethyl hexonate, sodium ethoxide and the like.

In still another embodiment of the present invention, the Ceftiofur sodium is isolated directly from the reaction mass comprising water miscible organic solvent and water. Most preferably precipitating Ceftiofur sodium from the reaction mass containing THF/water. The Ceftiofur sodium thus obtained was purified by either dissolving Ceftiofur sodium in water followed by isolating pure Ceftiofur sodium by adding sodium salt of mineral acid such as sodium chloride, or converting the Ceftiofur sodium into Ceftiofur TFA salt followed by converting Ceftiofur TFA salt into Ceftiofur sodium.

The starting material of the present invention can be prepared by utilizing the process available in the prior art.

The present invention is provided by the examples below, which are provided by way of illustration only and should not be considered to limit the scope of the invention.

### EXAMPLE 1

Preparation of Ceftiofur Sodium:

To a solution of 4-chloro-2-methoxyimino-3-oxobutyric acid (60.67 g) in dichloromethane (400 ml), phosphorus pentachloride (73.49 g) was added at −15 to −10° C. under nitrogen atmosphere. The reaction mass was stirred at −10 to −5° C. and washed with chilled purified water at 0-5° C. The organic layer was separated and added to a silylated solution of FURACA (prepared by treating suspension of FURACA (100 g) in dichloromethane (500 ml) with TMCS (24.52 g) and HMDS (36.4 g)) at −40 to −50° C.). After completion of reaction dichloromethane was distilled out under vacuum at 25-30° C. To the residue, aqueous THF (1000 mL) and thiourea (48 g) were added and stirred by maintaining pH at 4.0-8.0 using sodium bicarbonate at 10-20° C. After completion of the reaction, EDTA (5 g), sodium hydrosulphite (5 g) were added and cooled to 0-5° C. The solid obtained was filtered, washed with THF and dried under vacuum to yield pure title compound (107 g; purity by HPLC 99.28%).

### EXAMPLE 2

Preparation of Ceftiofur Sodium:

To the solution of 4-chloro-2-methoxyimino-3-oxobutyric acid (60.67 g) in dichloromethane (400 ml), phosphorus pentachloride (73.49 g) was added at −15 to −10° C. under nitrogen atmosphere. The reaction mass was stirred at −10 to −5° C. and washed with chilled purified water at 0-5° C. The organic layer was separated and added to a silylated solution of FURACA (prepared by treating suspension of FURACA (100 g) in dichloromethane (500 ml) with TMCS (24.52 g) and HMDS (36.4 g) at 10-20° C. and stirred to get clear solution at 25-30° C.) at −40 to −50° C. After completion of reaction dichloromethane was distilled out under vacuum at 25-30° C. To the residue THF (500 ml), DM water (500 ml) and thiourea (48 g) were added and stirred by maintaining pH at 5.0-8.0 using sodium bicarbonate at 18-22° C. To the reaction mixture was added sodium chloride (30 g) and separated aqueous layer. To the aqueous layer sodium chloride was added and stirred. The precipitated solid was filtered and washed with THF. Drying the solid under vacuum afforded pure title compound. (98 g, Purity by HPLC 98.48%).

### EXAMPLE 3

Preparation of Ceftiofur Sodium (without Silylating FURACA):

To the solution of 4-chloro-2-methoxyimino-3-oxobutyric acid (60.67 g) in dichloromethane (400 ml), phosphorus pentachloride (73.49 g) was added at −15 to −10° C. under nitrogen atmosphere. The reaction mass was stirred at −10 to −5° C. and washed with chilled purified water at 0-5° C. The organic layer was separated and added to a suspension of Furaca (100 g) in aqueous THF (20% & 1000 ml) by maintaining the pH at 5.5 to 8.5 using aqueous ammonia. To the reaction mixture was added thiourea (48 g) and the pH maintained in the range 5.0 to 8.0 using sodium bicarbonate. After completion of the reaction, THF was added to the reaction mass and cooled to 0° C. The solid obtained was filtered and washed with THF and dried under vacuum to yield pure title compound. (80 g; purity by HPLC 98.4 to 98.98).

### EXAMPLE 4

Preparation of Ceftiofur TFA Salt into Ceftiofur Sodium:

To the solution of Ceftiofur TFA salt in THF, triethylamine was added and adjusted the pH to 5.0-8.0. To the clear solution sodium 2-ethyl hexonate in THF was added at 0-25° C. The solid obtained was filtered and dried to get Ceftiofur sodium (99.7%) in pure form.

Preparation of Ceftiofur Sodium from Ceftiofur TFA salt:

To the solution of Ceftiofur TFA salt in THF, triethylamine was added and adjusted the pH to 5.0-8.0. To the clear solution sodium 2-ethylhexonate in THF was added at 0-25° C. The solid obtained was filtered and dried to get Ceftiofur sodium (purity 99.3 to 99.7%) in pure form.

What is claimed is:

1. An improved process for the preparation of Ceftiofur sodium of the formula (I) 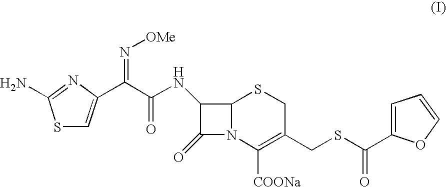
which comprises:
(i) activating the compound of formula (III) as acid chloride of formula (IIIa) in an organic solvent 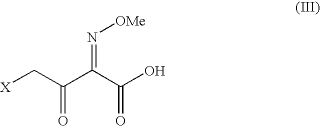
where X represents halogen atom such as chlorine or bromine, using an halogenating agent,
(ii) treating the reaction mass obtained from step (i) with water at a temperature in the range of −40° C. to 10° C.,
(iii) separating the organic layer containing the activated derivative of formula (IIIa) and condensing the activated derivative of the formula (IIIa) 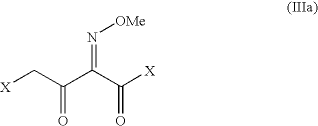
where X represents halogen atom such as chlorine or bromine, with 7-amino cephalosporin derivative of the formula (II) or silyl reactive derivative 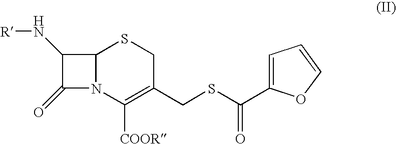
wherein R′ represents hydrogen, or silyl and R″ represents hydrogen or silyl in the presence of a solvent and in the presence or absence of base at a temperature in the range of −50° C. to 10° C. to produce a compound of formula (IV) 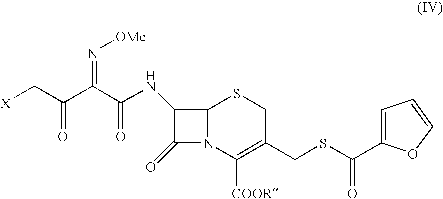
where all symbols are as defined above, and
iv) optionally removing the solvent of step (iii) reaction mass, and cyclizing the compound of formula (IV) with thiourea in the presence of water, in the presence or absence of water miscible solvent and sodium ion source, at a temperature in the range of −50 to 30° C. to produce compound of formula (I), wherein the improvement comprises producing the compound of formula (I) without isolating compound of formula (IV), and also characterized by one or more of the following:
a) removing the solvent in step (iii), and
b) conducting the reaction of step (iv) in a homogeneous solvent system.

2. The process as claimed in claim 1, wherein the organic solvent used in step (i) is dichloromethane, ethyl acetate, DMF, DMAc or mixtures thereof, and the solvent used for condensation in step (iii) is dichloromethane, ethyl acetate, methanol, ethanol, isopropanol, isobutyl alcohol, n-propanol, n-butanol, tert-butanol, tetrahydrofuran, aromatic hydrocarbons, acetone, ethyl methyl ketone, diethyl ketone, pentan-3-one, cyclohexanone, methyl isobutyl ketone, dioxane, acetonitrile, DMAc, N,N-dimethylformamide, dialkylethers, ethylene glycol, ethylene glycol monomethyl ether, diglyme, monoglyme, diethylene glycol, triethylene glycol, polyethylene glycol, water or mixtures thereof.

3. The process as claimed in claim 1, wherein the base used in step (iii) is ammonia, sodium carbonate, sodium bicarbonate, ammonium carbonate, barium carbonate, lithium carbonate, potassium carbonate, sodium hydroxide or potassium hydroxide, trimethyl amine, diisopropyl amine, diisopropyl ethylamine or mixtures thereof.

4. The process as claimed in claim 1, wherein the water miscible solvent used for cyclization is tetrahydrofuran, acetone, ethyl methyl ketone, methyl isobutyl ketone, methyl isopropyl ketone, cyclohexanone, diethyl ketone, pentan-3-one, cyclohexane, acetonitrile, N,N-dimethylformamide, N,N-dimethylacetamide, dioxane, (C₁-C₅)alcohol, ethylene glycol, diglyme, monoglyme, ethylene glycol monomethyl ether, diethylene glycol, triethylene glycol, polyethylene glycol or mixtures thereof.

5. The process as claimed in claim 1, wherein the water miscible solvent used for cyclization is tetrahydrofuran.

6. The process as claimed in claim 1, wherein the sodium ion source used in step (iii) is sodium carbonate, sodium bicarbonate, sodium hydroxide or sodium acetate.

7. The process as claimed in claim 1, wherein the reaction is carried out in a single pot.
